(19) United States
(12) Patent Application Publication
(10) Pub. No.: US 2015/0134145 A1
(43) Pub. Date: May 14, 2015

(54) METHOD AND APPARATUS FOR CONTROLLING MOVEMENT OF MEDICAL DEVICE

(71) Applicant: SAMSUNG ELECTRONICS CO., LTD., Suwon-si (KR)

(72) Inventors: Dong-jae LEE, Hwaseong-si (KR); Ho-seong KWAK, Seoul (KR)

(73) Assignee: SAMSUNG ELECTRONICS CO., LTD., Suwon-si (KR)

(21) Appl. No.: 14/461,765

(22) Filed: Aug. 18, 2014

(30) Foreign Application Priority Data
Nov. 8, 2013 (KR) 10-2013-0135846

(51) Int. Cl. A61B 19/00; G05D 1/02; G05D 1/00; A61B 17/00

(52) U.S. Cl. CPC A61B 19/5244; A61B 17/00; G05D 1/028; G05D 1/0016; A61B 2019/5255; A61B 2019/5231; A61B 2019/4857; A61B 2017/00225
USPC 701/2

(57) ABSTRACT

A method for controlling a movement of a medical device includes: acquiring location information of an object by using an ultra-wideband (UWB) location tracker; acquiring location information of the medical device by using a sensor disposed in the medical device; generating a movement path of the medical device based on the location information of the object and the location information of the medical device; and controlling the movement of the medical device based on the generated movement path.

### CROSS-REFERENCE TO RELATED APPLICATION

This application claims priority from Korean Patent Application No. 10-2013-0135846, filed on Nov. 8, 2013, in the Korean Intellectual Property Office, the disclosure of which is incorporated herein in its entirety by reference.

### BACKGROUND

1. Field

Methods and apparatuses consistent with exemplary embodiments relate to controlling a movement of a medical device by using an ultra-wideband (UWB).

2. Description of the Related Art

In general, when a medical device moves, an object may obstruct a movement path of the medical device. However, in the related art, the object is sensed only on a straight line with respect to the medical device. Therefore, there is a need for apparatuses and methods that allow a medical device to avoid an omnidirectional collision with an object.

### SUMMARY

One or more exemplary embodiments include methods and apparatuses for acquiring location information of an object and controlling a movement of a medical device by using ultra wideband (UWB).

According to one or more exemplary embodiments, a method for controlling a movement of a medical device by a control apparatus includes: acquiring location information of at least one object by using an UWB-based location tracker; acquiring location information of the medical device by using a sensor included in the medical device; generating a movement path of the medical device based on the location information of the at least one object and the location information of the medical device; and controlling the movement of the medical device based on the generated movement path.

The movement path of the medical device may include a movement path that allows the medical device to avoid a collision with the at least one object.

The acquiring of the location information of the at least one object may include acquiring the location information of the at least one object in a predetermined space by using the UWB-based location tracker.

The generating of the movement path may include: receiving target point information from a user; and generating the movement path in consideration of the received target point information.

The generating of the movement path may include: receiving, from a user, direction information about a direction in which the medical device is to move; and generating the movement path in consideration of the direction information.

The acquiring of the location information of the medical device may include acquiring the location information of the medical device by using at least one of an optical sensor and a magnetic sensor that are included in the medical device.

The acquiring of the location information of the medical device may include acquiring the location information of the medical device by using the location information of the at least one object.

The method may further include updating the location information of the at least one object by using the UWB-based location tracker.

The method may further include: updating the movement path by using the updated location information of the at least one object; and controlling the movement of the medical device based on the updated movement path.

According to one or more exemplary embodiments, an apparatus for controlling a movement of a medical device includes: a communicator configured to acquire location information of at least one object by using an UWB-based location tracker and acquire location information of the medical device by using a sensor included in the medical device; and a controller configured to generate a movement path of the medical device based on the location information of the at least one object and the location information of the medical device and control the movement of the medical device based on the generated movement path.

The movement path of the medical device may include a movement path that allows the medical device to avoid a collision with the at least one object.

The communicator may acquire the location information of the at least one object in a predetermined space by using the UWB-based location tracker.

The controller may receive target point information from a user and generate the movement path in consideration of the received target point information.

The apparatus may further include a user input unit configured to receive, from a user, direction information about a direction in which the medical device is to move, wherein the controller may generate the movement path in consideration of the direction information.

The communicator may acquire the location information of the medical device by using at least one of an optical sensor and a magnetic sensor that are included in the medical device.

The controller may acquire the location information of the medical device by using the location information of the at least one object.

The controller may update the location information of the at least one object by using the UWB-based location tracker.

The controller may update the movement path by using the updated location information of the at least one object and control the movement of the medical device based on the updated movement path.

The medical device may include an X-ray device.

According to one or more exemplary embodiments, a computer-readable recording medium stores a program that, when executed by a computer, performs the above-described method.

### BRIEF DESCRIPTION OF THE DRAWINGS

The above and/or other aspects will become more apparent by describing certain exemplary embodiments, with reference to the accompanying drawings, in which:

FIG. 1 is a diagram illustrating a control system for controlling a movement of a medical device, according to an exemplary embodiment;

FIG. 2 is a flowchart illustrating a method for controlling a movement of a medical device, according to an exemplary embodiment;

FIG. 3 is a diagram illustrating a method for locating an object by using a location tracker;

FIG. 4 is a diagram illustrating a method for analyzing location information of an object based on a signal received by a location tracker;

FIG. 5 is a diagram illustrating a method for updating a movement path of a medical device by tracking a location of a moving object;

FIG. 6 is a diagram illustrating a method for tracking a location of a medical device by using a sensor, according to an exemplary embodiment;

FIG. 7 is a diagram illustrating a method for generating a movement path of a medical device, according to an exemplary embodiment;

FIGS. 8A and 8B are diagrams illustrating a method for updating a movement path of a medical device, according to an exemplary embodiment;

FIGS. 9 and 10 are diagrams illustrating a method for controlling a movement of a medical device by using direction information, according to an exemplary embodiment;

FIG. 11 is a block diagram of an apparatus for controlling a movement of a medical device, according to an exemplary embodiment; and

FIG. 12 is a block diagram of an apparatus for controlling a movement of a medical device, according to an exemplary embodiment.

### DETAILED DESCRIPTION

Certain exemplary embodiments are described in greater detail below with reference to the accompanying drawings.

In the following description, the same drawing reference numerals are used for the same elements even in different drawings. The matters defined in the description, such as detailed construction and elements, are provided to assist in a comprehensive understanding of exemplary embodiments. Thus, it is apparent that exemplary embodiments can be carried out without those specifically defined matters. Also, well-known functions or constructions are not described in detail since they would obscure exemplary embodiments with unnecessary detail.

As used herein, expressions such as “at least one of,” when preceding a list of elements, modify the entire list of elements and do not modify the individual elements of the list.

The terms used in this specification are those general terms currently widely used in the art in consideration of functions in regard to the present teaching, but the terms may vary according to the intention of those of ordinary skill in the art, precedents, or new technology in the art. Also, specified terms may be selected by the applicant, and the detailed meaning thereof will be described in the detailed description. Thus, the terms used in the specification should be understood not as simple names but based on the meaning of the terms and the overall description.

When something “comprises” or “includes” a component, another component may be further included unless specified otherwise. Also, the term “unit” used herein refers to a software component or a hardware component such as a field-programmable gate array (FPGA) or an application-specific integrated circuit (ASIC), and the “unit” performs some functions. However, the “unit” is not limited to software or hardware. The “unit” may be formed so as to be in a addressable storage medium, or may be formed so as to operate one or more processors. Thus, for example, the “unit” may include components such as software components, object-oriented software components, class components, and task components, and may include processes, functions, attributes, procedures, subroutines, segments of program code, drivers, firmware, micro codes, circuits, data, a database, data structures, tables, arrays, and variables. A function provided by the components and “units” may be associated with the smaller number of components and “units”, or may be divided into additional components and “units”.

In the specification, a “user” may be, but is not limited to, a doctor, a nurse, a medical laboratory technologist, a medial image expert, a radiologist, a care taker, and a technician who repairs a medical apparatus.

In the specification, the UWB refers to a radio technology for transmitting a large amount of digital data within a short range with low power through a wide spectrum frequency band. However, exemplary embodiments are not limited to the UWB.

UWB may be performed with low-power pulses of thousands to millions of times per second while using GHz-band frequencies.

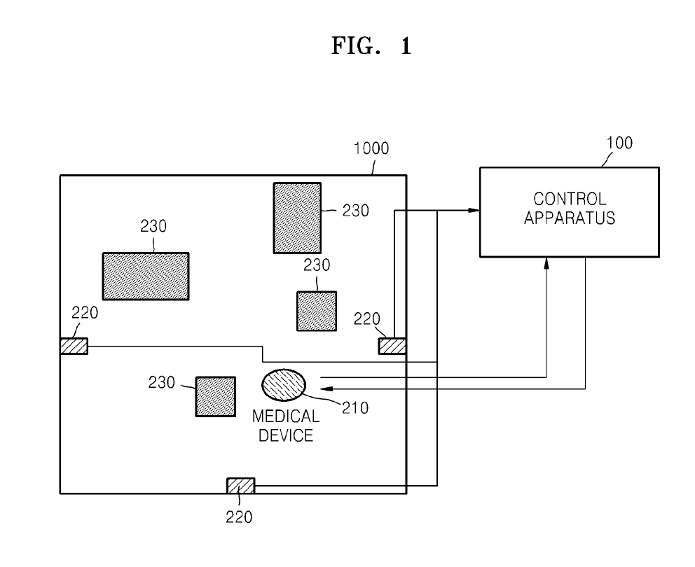

FIG. 1 is a diagram illustrating a control system for controlling a movement of a medical device, according to an exemplary embodiment.

As illustrated in FIG. 1, the control system according to an exemplary embodiment may include a control apparatus 100, a medical device 210, and one or more location trackers 220, for example, UWB-based location trackers.

The control apparatus 100 may be an apparatus for controlling a movement of the medical device 210. The control apparatus 100 may acquire location information of at least one object 230 that obstructs a movement of the medical device 210, by using a location tracker 220. The location tracker 220 may be attached on a wall, a ceiling, or a floor. The location tracker 220 may include an antenna for receiving signals, for example, UWB signals and an analyzing module for analyzing received signals. The location tracker 220 may include unique identification information (ID).

The control apparatus 100 may control a movement of the medical device 210 by using the location information of the object that is acquired by using the location tracker 220. The object 230 may be a living object or a nonliving object. For example, the object 230 may include, but is not limited to, a medical device, a wall, a human, an animal, and a plant. According to an exemplary embodiment, the object 230 may be fixed or movable.

The control apparatus 100 may communicate with the medical device 210, in a wired or wireless manner. For example, the control apparatus 100 may control the movement of the medical device 210 by various communication methods. The control apparatus 100 may receive current location information of the medical device 210 from the medical device 210. The control apparatus 100 may generate a movement path, through which the medical device 210 is to move, by using the location information of the medical device 210 and the location information of the object 230. The control apparatus 100 may transmit a control signal, including information about the movement path, to the medical device 210.

The control apparatus 100 may exchange data with a hospital server or other medical apparatuses in a hospital connected via a picture archiving and communication system (PACS).

The control apparatus 100 may include a touchscreen. The touchscreen may be configured to detect a touch input pressure, a touch input position, and/or a touch area. The touchscreen may be configured to detect a proximity touch as well as a real touch.

The direct touch refers to a case where a user touches a screen with a touch tool (e.g., a finger or an electronic fen), and the proximity touch refers to a case where a user approaches a touch tool to a screen within a predetermined distance without touching the screen.

The medical device 210 may be an apparatus that acquires a medical image of an object. For example, the medical device 210 may include, but is not limited to, an X-ray device.

According to an exemplary embodiment, the medical device 210 may be movable. For example, the medical device 210 may move by using a rail that is attached on a ceiling. The medical device 210 may move to another place based on a control signal received from the control apparatus 100.

According to an exemplary embodiment, the medical device 210 may acquire location information of the medical device 210 by using a sensor. The medical device 210 may transmit the location information of the medical device 210 to the control apparatus 100. According to an exemplary embodiment, the medical device 210 may transmit the location information of the medical device 210 by various communication methods.

For example, the medical device 210 may use, but is not limited to, wired communication, wireless communication, or short-range wireless communication.

Examples of short-range communication technologies may include, but are not limited to, wireless local area network (LAN), Wi-Fi, Bluetooth, Zigbee, Wi-Fi Direct (WFD), infrared data association (IrDA), Bluetooth Low Energy (BLE), and near field communication (NFC).

According to an exemplary embodiment, at least one medical device 210, the location tracker 220, and the object 230 may be located in a space 1000, i.e., a room. The predetermined space may be a closed space. The predetermined space may be a region in which the rail used to move the medical device 210 is located.

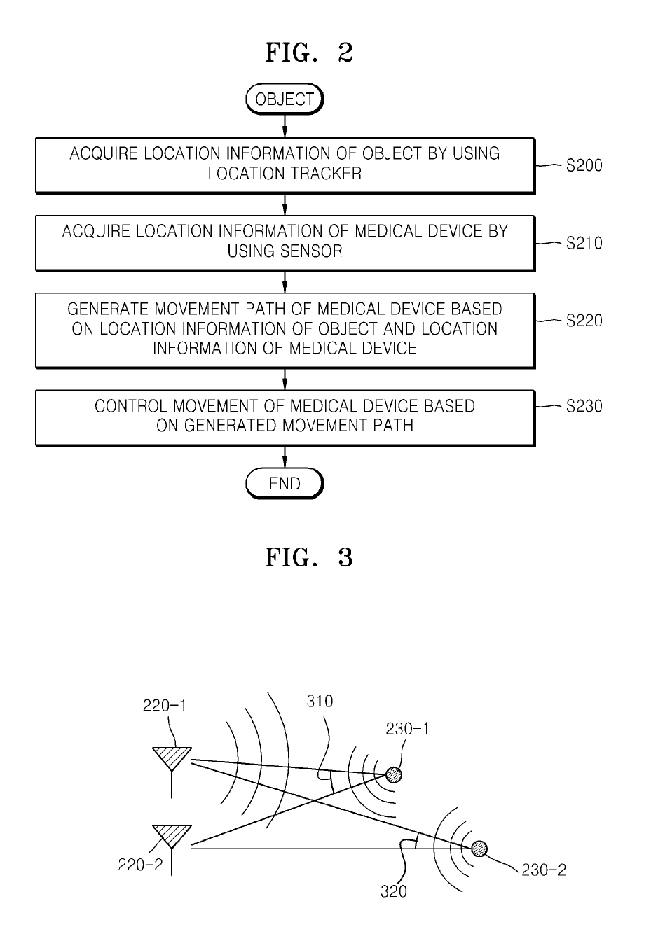

A method for the control apparatus 200 to control a movement of the medical device 210 by using UWB is described in detail below with reference to FIG. 2.

FIG. 2 is a flowchart illustrating a method for controlling a movement of a medical device, according to an exemplary embodiment.

In operation S200, the control apparatus 100 may acquire location information of at least one object 230 by using the location tracker 220. According to an exemplary embodiment, the location information of at least one object 230 may include information about one or more coordinates of at least one object 230 in a closed space. The location information of at least one object 230 may include information about an angle between the object 230 and the medical device 210, and direction information of the object 230.

The control apparatus 100 may acquire location information of the object 230 by using at least two location trackers 220. For example, the control apparatus 100 may receive information about a signal reflected by the object 230, from each of two location trackers 220. The information about the reflected signal may include information about a reception angle of the reflected signal, and information about a reception time of the reflected signal.

The location tracker 220 may include a tracking device that transmits a UWB pulse and a sensor that receives and calculates a signal received from the tracking device. The sensor may sense a low-power UWB pulse, and may discriminate between a directly-received signal and a reflected received signal.

By using two location trackers 220, the control apparatus 100 may track a location of an object based on a distance between the object and each location tracker 220 and an angle of arrival (AoA) obtained from two or more location trackers 220. The control apparatus 100 may acquire a time difference of arrival (TDoA) from each location tracker 220 by using two or more location trackers 220. The control apparatus 100 may acquire location information of the object 230 by tracking a point at which the TDoA from each location tracker 220 is constant.

An operation of the control apparatus 100 for acquiring the location information of the object 230 is described below in detail with reference to FIGS. 3 to 5.

In operation S210, the control apparatus 100 may acquire location information of the medical device 210 by using a sensor included in the medical device 210.

The sensor included in the medical device 210 may include at least one of an optical sensor and a magnetic sensor. The medical device 210 may move along an installed rail. The control apparatus 100 may acquire location information of the medical device 210 on the installed rail by using the sensor included in the medical device 210.

For example, the installed rail may have a light-transmissible region and a light non-transmissible region, and the medical device 210 may acquire current location information of the medical device 210 by using the optical sensor. Also, when there are an N-pole region and an S-pole region in the installed rail, the medical device 210 may obtain location information of the medical device 210 by using the magnetic sensor.

The optical sensor may acquire the location information of the medical device 210 by sensing light and discriminating between a light-transmitting portion and a light non-transmitting portion. The magnetic sensor may acquire the location information of the medical device 210 by discriminating between an N pole and an S pole by using a magnetic field.

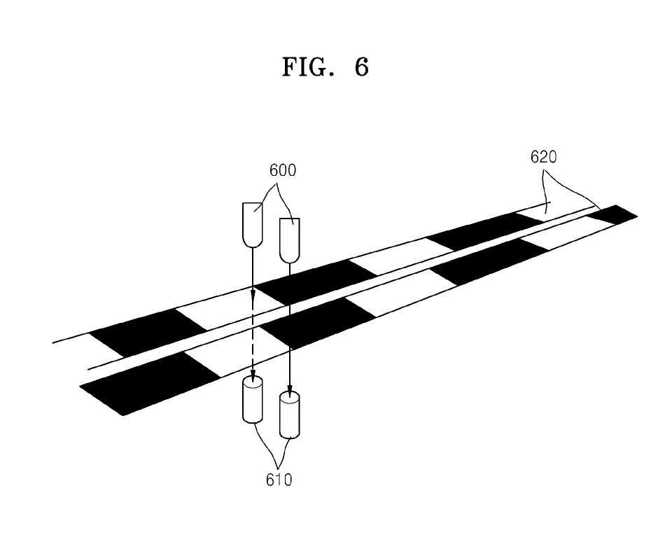

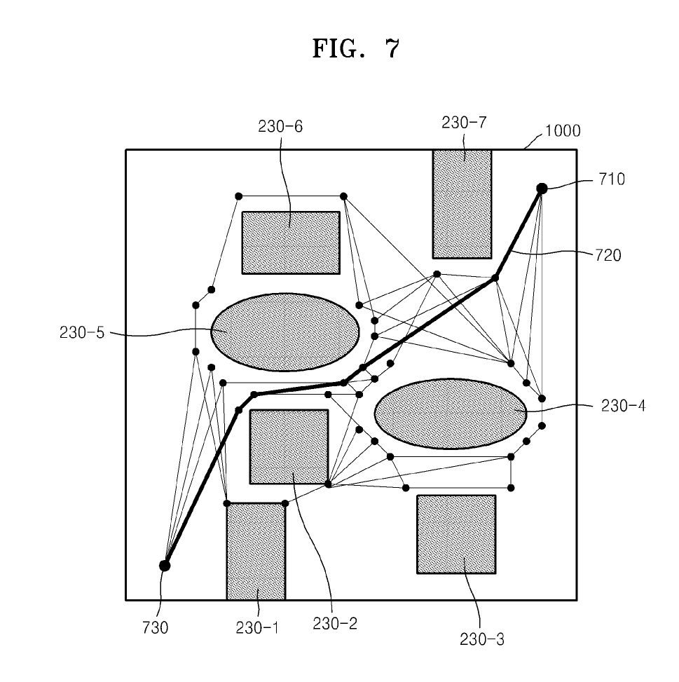

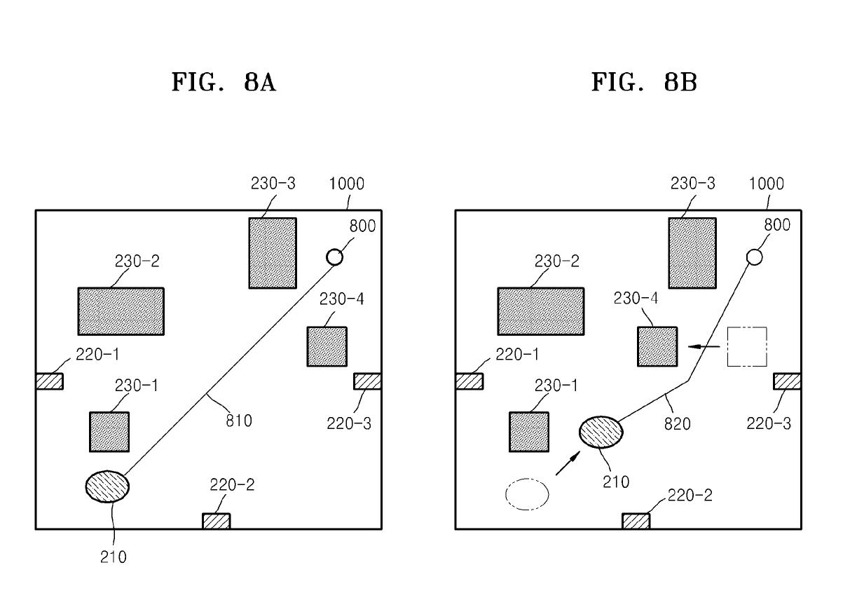

The sensor included in the medical device 210 is described below in detail with reference to FIG. 6.

In operation S220, the control apparatus 100 may generate a movement path of the medical device 210 based on the location information of the object 230 and the location information of the medical device 210. The movement path of the medical device 210 may include a movement path that the medical device 210 to avoid a collision with the object 230. When the medical device 210 avoids a collision with an object, there is no direct contact between the medical device 210 and a portion of the object 230. The control apparatus 100 may generate a movement path for avoiding a collision with the object 230, by using the size of the medical device 210 and the location information of the medical device 210 that are input in advance.

The control apparatus 100 may detect a location at which the movement of the medical device 210 is obstructed, based on the location information of the object 230. For example, the control apparatus 100 may compare the locations of the medical device 210 and other objects by using the location information of the medical device 210.

The control apparatus 100 may receive target point information according to a user input. The control apparatus 100 may generate a movement path from the location of the medical device 210 to a target point. The target point information may be information about a location to which the user is to move the medical device 210. For example, the target point information may include coordinates of the target point, a direction with respect to the target point, or an angle of the medical device 210 with respect to the target point.

The control apparatus 100 may generate a shortest movement path that allows the medical device 210 to reach the target point while avoiding a collision with the object 230. The control apparatus 100 may receive direction information about a direction in which the medical device 210 is to move, according to a user input. The direction information may be any one of the front, back, left, and right directions of the medical device 210.

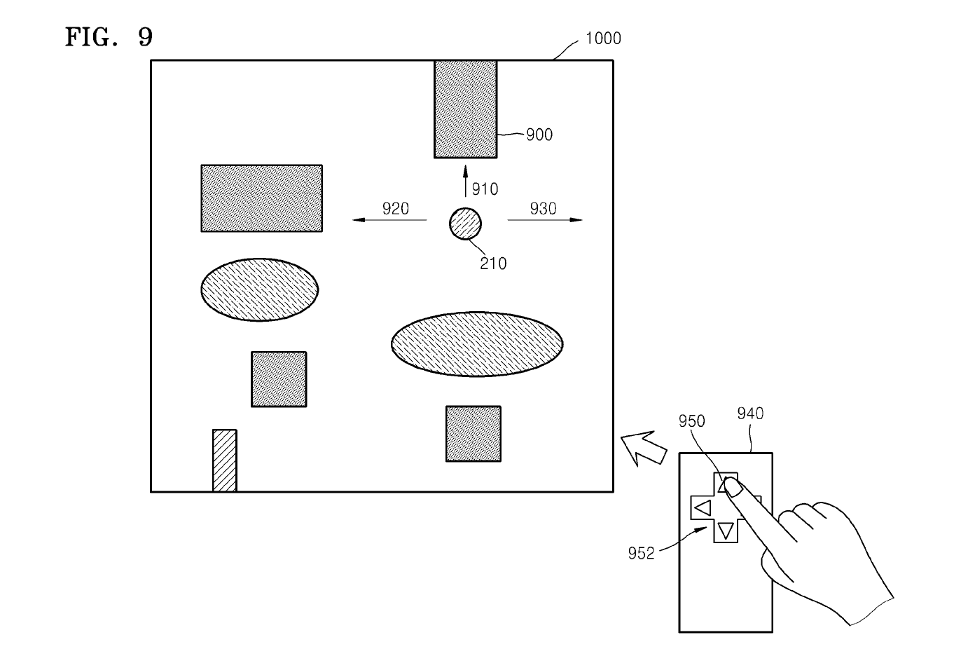

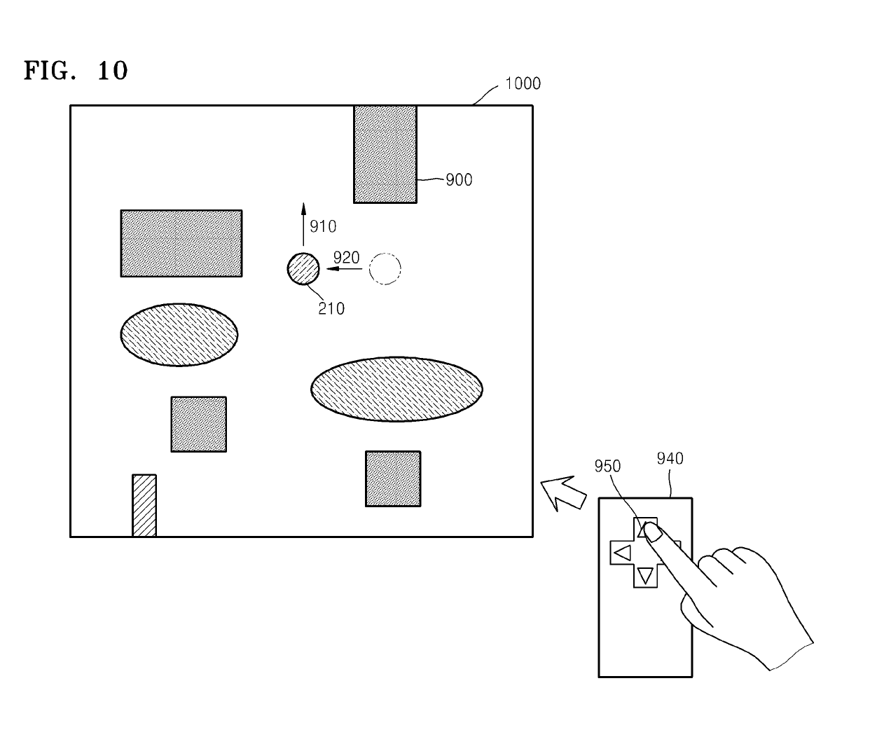

The control apparatus 100 may generate a movement path in consideration of the direction in which the medical device 210 is to move. For example, the control apparatus 100 may include a motor and a driver (not illustrated) configured to move the medical device along the generated movement path. A method of generating the movement path is described below in detail with reference to FIGS. 9 and 10.

In operation S230, the control apparatus 100 may control the movement of the medical device 210 based on the generated movement path.

For example, the control apparatus 100 may transmit a movement control signal that allows the medical device 210 to move along the path from the current point to the target point input by the user. The control apparatus 100 may transmit the movement control signal to the medical device 210 by using wired or wireless communication.

FIG. 3 is a diagram illustrating a method for locating an object by using a UWB-based location tracker.

Referring to FIG. 3, the control apparatus 100 may receive a first signal from a first location tracker 220-1. The first signal may be a signal that is reflected from a first object 230-1 and is acquired by the first location tracker 220-1. The control apparatus 100 may receive a second signal from a second location tracker 220-2. The second signal may be a signal that is reflected from the first object 230-1 and is acquired by the second location tracker 220-2. The control apparatus 100 may acquire information about the distance and an angle 310 between the first object 230-1 and the first and second location trackers 220-1 and 220-2 by using the first signal and the second signal. The control apparatus 100 may acquire location information of the first object 230-1 by using the information about the angle 310 and the distance.

The control apparatus 100 may receive a third signal from the first location tracker 220-1. The third signal may be a signal that is reflected from a second object 230-2 and is acquired by the first location tracker 220-1. The control apparatus 100 may receive a fourth signal from the second location tracker 220-2. The fourth signal may be a signal that is reflected from the second object 230-2 and is acquired by the second location tracker 220-2. The control apparatus 100 may acquire information about the distance and an angle 320 between the second object 230-2 and the first and second location trackers 220-1 and 220-2 by using the third signal and the fourth signal. The control apparatus 100 may acquire location information of the second object 230-2 by using the information about the angle 320 and the distance.

In FIG. 3, the first location tracker 220-1 and the second location tracker 220-2 are shown as being disposed on the same line, but this is illustrative only and not limiting. The first location tracker 220-1 and the second location tracker 220-2 may be disposed at any location in the space 1000.

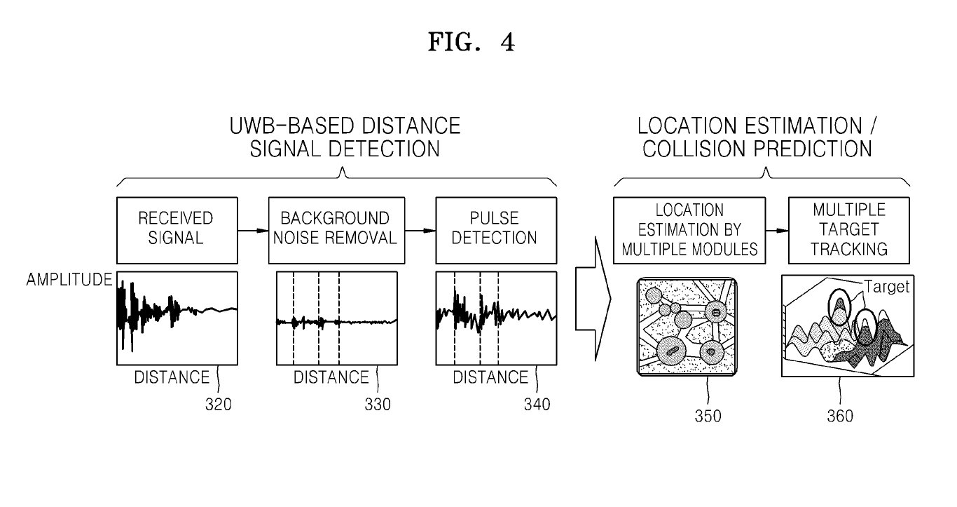

FIG. 4 is a diagram illustrating a method for analyzing location information of an object based on a signal received by a UWB-based location tracker.

Referring to FIG. 4, the location tracker 220 may receive a signal through an antenna and estimate a location of the object 230 based on the received signal.

Graph 320 illustrates a signal that is received by the location tracker 220 through the antenna. The location tracker 220 may receive a signal that is generated when a signal transmitted by the location tracker 220 is reflected by the object 230. The signal received by the location tracker 220 may include noise and a signal that is generated when the signal transmitted by the location tracker 220 is reflected by the object 230. The amplitude of the signal may decrease as the distance to the object increases.

Graph 330 illustrates a signal that is obtained by removing a noise from a received signal. The control apparatus 100 may remove a noise from the received signal by removing a predetermined frequency and amplitude of the noise.

Graph 340 illustrates a pulse that is generated when a noise-removed signal is reflected by the object 230. The control apparatus 100 may detect the pulse by comparing a transmitted signal and a received signal with respect to the noise-removed signal. The control apparatus 100 may detect a location of the object 230 and a distance from the object 230 based on the detected pulse. The control apparatus 100 may detect an angle between the object 230 and the location tracker 220 based on a pulse that is detected by a plurality of location trackers 220.

Reference numeral 350 is a diagram illustrating the estimation of the location of at least one object 230 by using a plurality of location trackers 220. A method of estimating the location of the object 230 is the same as described with reference to FIG. 3. When there are two or more location trackers 220, the control apparatus 100 may detect a one-dimensional (1D) location of the object. When there are three or more location trackers 220, the control apparatus 100 may detect a two-dimensional (2D) location of the object. When there are four or more location trackers 220, the control apparatus 100 may detect a three-dimensional (3D) location of the object.

Reference numeral 360 is a diagram illustrating the tracking of the locations of a plurality of objects 230 in real time. The control apparatus 100 may track the location of an object 230, i.e., a target, by updating the location of the object 230 in real time.

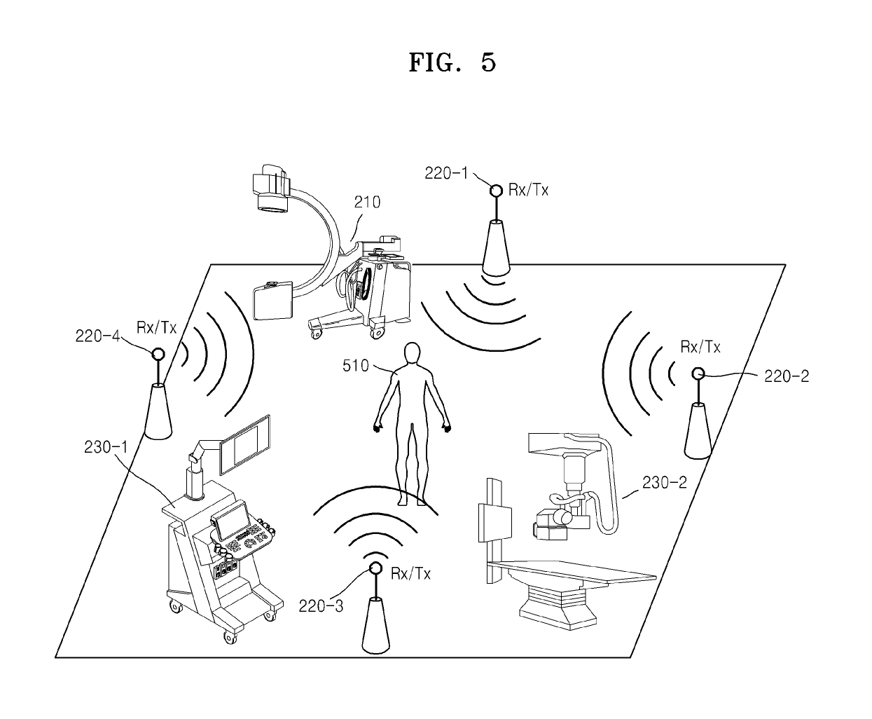

FIG. 5 is a diagram illustrating a method for updating a movement path of a medical device by tracking a location of a moving object by using UWB.

The control apparatus 100 may track a location of a moving object 510 by using a first location tracker 220-1, a second location tracker 220-2, a third location tracker 220-3, and a fourth location tracker 220-4. The control apparatus 100 may track a location of the medical device 210, a location of the first object 230-1, a location of the second object 230-2, and a location of the moving object 510.

The control apparatus 100 may generate a movement path of the medical device 210 to a target point in consideration of the movement of the moving object 510. The control apparatus 100 may control the movement of the medical device 210 based on the updated movement path.

FIG. 6 is a diagram illustrating a method for tracking a location of a medical device by using a sensor, according to an exemplary embodiment.

Referring to FIG. 6, the optical sensor includes a light source 600 and a light receiving device 610 that face each other with a scale 620 interposed therebetween. A black portion and a white portion appear alternately in the scale 620. Light emitted from the light source 600 is irradiated onto the light receiving device 610. The control apparatus 100 may track the location of the medical device 210 spaced apart from a reference location, based on information about whether the light emitted from the light source 600 is sensed by the light receiving device 610. For example, an encoder may be used to track the location of the medical device 210. The encoder may include an optical sensor, a light receiving device 610, and a scale 620.

For example, the control apparatus 100 processes a signal as 1 when light is sensed, processes a signal as 0 when light is not sensed, and assumes that a black portion (non-transmissible portion) and a white portion (transmissible portion) appear alternately every 1 cm. A signal may be processed as 1, 0, 1, when the medical device 210 moves, and the control apparatus 100 may acquire information indicating that the medical device 210 has moved through a white portion twice and has moved through a black portion once. In this case, the control apparatus 100 may acquire information indicating that the medical device 210 is spaced apart from the reference location by about 3 cm. The sensor may use a magnetic sensor as well as the optical sensor. The magnetic sensor may track a location by sensing whether a pole is an N pole or an S pole, instead of sensing light.

FIG. 7 is a diagram illustrating a method for generating a movement path of a medical device, according to an exemplary embodiment.

Referring to FIG. 7, the control apparatus 100 generates a movement path from a start point 730, at which the medical device 210 is located, to a target point 710 according to a user input. A plurality of objects may be placed in a space 1000.

In general, the medical device 210 may track only the object 230 on a straight line through the sensor. By using the exemplary location tracker 220, the control apparatus 100 may acquire location information of all of the objects in the space 1000. The control apparatus 100 may acquire location information of the medical device 210 by using a sensor installed at the medical device 210. By using the locations of the objects, the control apparatus 100 may generate a movement path that allows the medical device 210 to move from a start point 730 to a target point 710 while avoiding a collision with objects 230-1, 230-2, 230-3, 230-4, 230-5, 230-6, and 230-7.

The control apparatus 100 may generate the movement path in consideration of the size of the medical device 210. The control apparatus 100 may generate the movement path based on the assumption that an object 230 is larger than by a predetermined size than the actual size of the object 230 acquired by the location tracker 220. The predetermined size may be the size of the medical device 210, i.e., to ensure clearance for the approach of the medical device 210. The control apparatus 100 may select a shortest movement path 720 among a plurality of movement paths and control the movement of the medical device 210 based on the selected movement path.

FIGS. 8A and 8B are diagrams illustrating a method for updating a movement path of the medical device 210, according to an exemplary embodiment.

FIGS. 8A and 8B illustrate a method for the control apparatus 100 to update a movement path of the medical device 210 by updating the locations of the first to fourth objects 230-1, 230-2, 230-3, and 230-4 placed in the space 1000 by using the location trackers 220-1, 220-2, and 220-3.

Referring to FIG. 8A, the control apparatus 100 may transmit a control signal, which allows the medical device 210 to move through a shortest movement path 810 to a target point 800 input by the user while avoiding a collision with the objects, to the medical device 210.

Referring to FIG. 8B, the fourth object 230-4 may obstruct the movement path of the medical device 210 by moving to another location while the medical device 210 moves. The location trackers 220-1, 220-2, and 220-3 may acquire location of the objects at predetermined periods. The control apparatus 100 may update the previous movement path based on the acquired location information. If the medical device 210 moves along the previously determined movement path, the medical device 210 may collide with the object 230-4. On the other hand, when the medical device 210 moves along an updated movement path 820, the medical device 210 may reach the target point 800 without colliding with the object 230-4.

FIGS. 9 and 10 are diagrams illustrating a method for controlling a movement of the medical device 210, according to an exemplary embodiment.

The medical device 210 and the object 900 may be located in a space 1000. The control apparatus 100 may receive direction information 950 about a direction 910 in which the medical device 210 is to move, through a remote controller 940, by operation of a directional key 952 or other keys provided in the remote controller 940. Based on the received direction information 950, the control apparatus 100 may perform control so that the medical device 210 moves in the direction 910. When the object 900 is located on a path in the direction 910 of the medical device 210, the control apparatus 100 may perform control so that the medical device 210 moves to the left 920 of the object 900 or to the right 930 of the object 900.

In FIG. 10, the medical device 210 may move to the left 920 of the object 900 in order to avoid a collision with the object 900. When the direction information 950 about the direction 910 of the medical device 210 is not input for a predetermined time, the control apparatus 100 may perform control so that the medical device 210 moves in the direction 910. Thus, the control apparatus 100 may perform control that the medical device 210 that is moving to the left 920 of the object 900 moves in the direction 910 again.

FIG. 11 is a block diagram of an apparatus for controlling a movement of a medical device, according to an exemplary embodiment.

Referring to FIG. 11, a control apparatus 100 controlling a movement of a medical device 210 may include a communicator 110 and a controller 120.

The communicator 110 may acquire location information of at least one object 230 by using a UWB-based location tracker. The communicator 110 may acquire location information of the medical device 210 by using a sensor included in the medical device 210. The communicator 110 may communicate with the location tracker in a wired or wireless manner. The communicator 110 may communicate with a server and may also communicate with a user interface. For example, the communicator 110 may include a short-range wireless communicator. Short-range communication technologies may include, but are not limited to, wireless local area network (LAN), Wi-Fi, Bluetooth, Zigbee, Wi-Fi Direct (WFD), UWB, infrared data association (IrDA), Bluetooth Low Energy (BLE), and near field communication (NFC).

Bluetooth is a standard for performing wireless communication between wireless communication devices within a short range with low power. UWB is a radio technology for a large amount of digital data within a short range with low power through a wide spectrum frequency band.

WFD is a new version of Wi-Fi and enables direct communication between devices. That is, WFD devices may communicate and share information with each other even without using a hot spot, a router, or an access point (AP).

ZigBee is one of the IEEE 802.15.4 standards that support short-range wireless communication. ZigBee is a technology for ubiquitous computing and wireless communication within a distance of about 10 m to about 20 m in wireless network fields of a home, an office, or the like.

BLE is one of the wireless communication technologies and refers to a core function of Bluetooth v 4.0. BLE has a lower duty cycle and may be implemented at a lower price than the classic Bluetooth standards, and a BLE device needs low average power and low standby power and thus may operate for several years with a coin-size battery.

NFC is a type of electronic tag (RFID), and may be a contactless NFC module using a frequency band of about 13.56 MHz. NFC may be used to transmit data between terminals within a short distance of about 10 cm.

The controller 120 may generate a movement path of the medical device 210 based on the location information of the object 230 and the location information of the medical device 210. The controller 120 may control the movement of the medical device 210 based on the generated movement path.

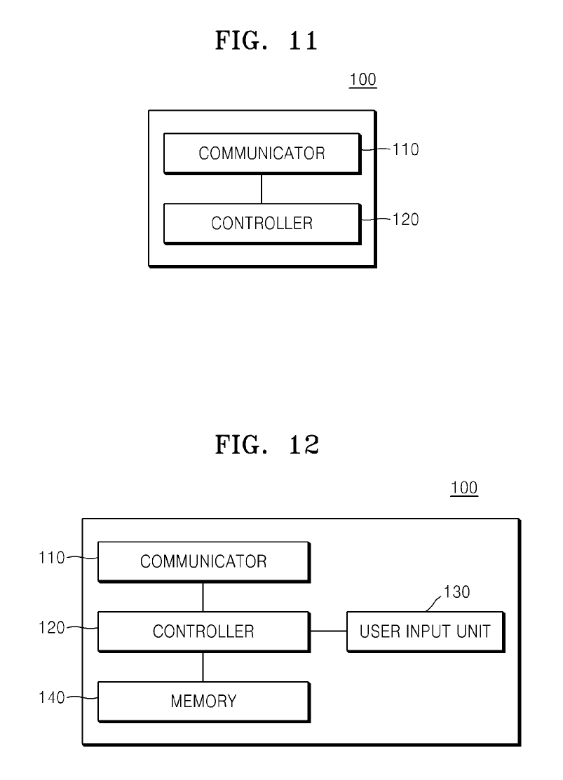

FIG. 12 is a block diagram of an apparatus for controlling a movement of a medical device, according to an exemplary embodiment.

Referring to FIG. 12, a control apparatus 100 controlling a movement of a medical device 210 may include a communicator 110, a controller 120, a user input unit 130, and a memory 140.

The communicator 110 and the controller 120 are the same as those described with reference to FIG. 11.

The user input unit 130 may receive target point information from a user. The user input unit 130 may receive, from the user, direction information about a direction in which the medical device 210 is to move. The user input unit 130 may be a remote controller. The user input unit 130 may be a touchscreen type that may touch and input information displayed on a display.

The memory 140 may store at least one of the location information of the object 230 that is acquired by using the UWB location tracker 220, and the location information of the medical device 210 that is acquired by using a sensor included in the medical device 210. The memory may store the target point information received through the user input unit 130, or the direction information about a direction in which the medical device 210 is to move. The memory 140 may store a program for processing and control of the controller 120, and may store input/output data (for example, information for controlling the movement of the medical device 210). For, example the memory 140 may include at least one storage medium including at least one of a flash memory, hard disk, multimedia card (MMC) micro, card memory (e.g., SD and XD memories), random access memory (RAM), static random access memory (SRAM), read-only memory (ROM), electronically erasable programmable read-only memory (EEPROM), programmable read-only memory (PROM), magnetic memory, magnetic disk, and optical disk. The control apparatus 100 may operate a cloud server or a web storage that performs a storage function of the memory 140 on the Internet.

The control apparatus 100 may display programs, which are stored in the memory 140, on the display.

The exemplary embodiments may be written as computer programs and may be implemented in general-use digital computers that execute the programs using a computer-readable recording medium.

Examples of the computer-readable recording medium include magnetic storage media (e.g., ROM, floppy disks, hard disks, etc.), optical recording media (e.g., CD-ROMs, DVDs, etc.), and transmission media such as Internet transmission media.

The described-above exemplary embodiments and advantages are merely exemplary and are not to be construed as limiting. The present teaching can be readily applied to other types of apparatuses. The description of exemplary embodiments is intended to be illustrative, and not to limit the scope of the claims, and many alternatives, modifications, and variations will be apparent to those skilled in the art.

What is claimed is:

1. A method for controlling a movement of a medical device, the method comprising:
acquiring an object location information of an object by using an ultra-wideband (UWB) location tracker;
acquiring a device location information of the medical device by using a sensor disposed in the medical device;
generating a movement path of the medical device based on the object location information and the device location information; and
controlling the movement of the medical device based on the generated movement path.

2. The method of claim 1, wherein the movement path of the medical device comprises a movement path that allows the medical device to avoid a collision with the object.

3. The method of claim 1, wherein the acquiring the object location information comprises:
acquiring the object location information in a predetermined space.

4. The method of claim 1, wherein the generating the movement path comprises:
receiving target point information from a user; and
generating the movement path based on the received target point information.

5. The method of claim 1, wherein the generating the movement path comprises:
receiving, from a user, direction information about a direction in which the medical device is to move; and
generating the movement path based on the direction information.

6. The method of claim 1, wherein the acquiring the device location information comprises:
acquiring the device location information by using at least one of an optical sensor and a magnetic sensor that are disposed in the medical device.

7. The method of claim 1, wherein the acquiring the device location information comprises:
acquiring the device location information by using the object location information.

8. The method of claim 1, further comprising updating the object location information by using the UWB location tracker.

9. The method of claim 8, further comprising:
updating the movement path by using the updated object location information; and
controlling the movement of the medical device based on the updated movement path.

10. An apparatus for controlling a movement of a medical device, the apparatus comprising:
a communicator configured to acquire object location information of an object by using an ultra-wideband (UWB) location tracker and acquire device location information of the medical device by using a sensor disposed in the medical device; and
a controller configured to generate a movement path of the medical device based on the object location information and the device location information and control the movement of the medical device based on the generated movement path.

11. The apparatus of claim 10, wherein the movement path of the medical device comprises a movement path that allows the medical device to avoid a collision with the object.

12. The apparatus of claim 10, wherein the communicator acquires the location information of the object in a predetermined space.

13. The apparatus of claim 10, wherein the controller receives target point information from a user and generates the movement path based on the received target point information.

14. The apparatus of claim 10, further comprising:
a user input unit configured to receive, from a user, direction information about a direction in which the medical device is to move,
wherein the controller generates the movement path based on the direction information.

15. The apparatus of claim 10, wherein the communicator acquires the device location information by using at least one of an optical sensor and a magnetic sensor disposed in the medical device.

16. The apparatus of claim 10, wherein the controller calculates the device location information by using the object location information.

17. The apparatus of claim 10, wherein the controller updates the object location information by using the UWB-based location tracker.

18. The apparatus of claim 17, wherein the controller updates the movement path by using the updated object location information and controls the movement of the medical device based on the updated movement path.

19. The apparatus of claim 10, wherein the medical device comprises an X-ray device.

20. An apparatus for controlling a movement of a medical device, the apparatus comprising:
location trackers which are disposed at different locations inside an area and configured to detect locations of objects disposed in the area;
a sensor which is installed in a vicinity of the medical device and configured to detect a location of the medical device;
a processor configured to generate a movement path of the medical device to a target point, within the area, based on the detected locations of the objects and the detected location of the medical device; and
a driver configured to move the medical device along the generated movement path.

21. The apparatus of claim 20, wherein the location trackers are configured to monitor a change in the locations of the objects,
the processor is configured to generate a new movement path of the medical device to the target point within the area, based on the change in the locations of the objects and a current location of the medical device; and
the driver is configured to move the medical device along the new movement path that allows the medical device to avoid a collision with the objects.

22. The apparatus of claim 20, wherein the controller receives an input of the target point from a remote controller.

23. The apparatus of claim 20, wherein the sensor comprises at least one of an optical sensor and a magnetic sensor and the location trackers comprise an ultra-wide band trackers.

24. The apparatus of claim 20, wherein the medical device comprises a mobile X-ray apparatus.
